(19) United States
(12) Patent Application Publication
(10) Pub. No.: US 2025/0019687 A1
(43) Pub. Date: Jan. 16, 2025

(54) MICROFLUIDIC CHIP, NUCLEIC ACID EXTRACTION APPARATUS AND NUCLEIC ACID EXTRACTION METHOD

(71) Applicants: Beijing BOE Technology Development Co., Ltd., Beijing (CN); BOE Technology Group Co., Ltd., Beijing (CN)

(72) Inventors: Beiyuan FAN, Beijing (CN); Ding DING, Beijing (CN)

(21) Appl. No.: 18/274,203

(22) PCT Filed: May 31, 2022
(86) PCT No.: PCT/CN2022/096119

§ 371 Date: Jul. 25, 2023

(51) Int. Cl. C12N 15/10

(52) U.S. Cl. CPC C12N 15/1013

(57) ABSTRACT

The present disclosure provides a microfluidic chip configured to extract a nucleic acid, the microfluidic chip including: a first substrate having at least two buffer areas configured to cooperate with a magnet, and being provided with a microfluidic channel, wherein the microfluidic channel includes a primary flow channel and a plurality of buffer flow channels connected in series to the primary flow channel, the buffer flow channels are in the buffer areas, different buffer flow channels are in different buffer areas, respectively, and each of the buffer flow channels has a double-spiral shape; and a second substrate opposite to the first substrate. The microfluidic chip has a plurality of openings in communication with the primary flow channel. The present disclosure further provides a nucleic acid extraction apparatus and a nucleic acid extraction method.

### CROSS REFERENCE TO RELATED APPLICATIONS

This is a National Phase Application filed under 35 U.S.C. 371 as a national stage of PCT/CN2022/096119 filed on May 31, 2022, the entire content of which is incorporated herein by reference.

### TECHNICAL FIELD

The present disclosure relates to the field of biotechnology, and in particular, to a microfluidic chip, a nucleic acid extraction apparatus, and a nucleic acid extraction method.

### BACKGROUND

The microfluidic technology, which is a new scientific technology, has been applied to various fields such as chemistry, biology, engineering, physics, and the like, involves a complex interdisciplinary, makes breakthroughs in precise controls of time, space and an analysis object, and can solve many key problems of life analysis. The microfluidic technology can integrate detection experiments, which can only be completed in a laboratory in the past, on a small chip, such that the material cost and the time cost are saved, more importantly, various detection technologies can be integrated into a whole, and a detection efficiency is improved.

Microfluidic chips are widely applied to the field of biochemical analysis, such as the field of nucleic acid detection, and the like, due to their characteristics of easy integration, automation, controllable fluid and small required sample volume, and overcome the disadvantage that a professional nucleic acid extraction apparatus is difficult to deploy in a non-laboratory area to a certain extent.

### SUMMARY

The present disclosure provides a microfluidic chip, a nucleic acid extraction apparatus, and a nucleic acid extraction method.

The present disclosure provides a microfluidic chip configured to extract a nucleic acid, the microfluidic chip including:
a first substrate having at least two buffer areas configured to cooperate with a magnet, and being provided with a microfluidic channel, wherein the microfluidic channel includes a primary flow channel and a plurality of buffer flow channels connected in series to the primary flow channel, the buffer flow channels are in the buffer areas, different buffer flow channels are in different buffer areas, respectively, and each of the buffer flow channels has a double-spiral shape; and
a second substrate opposite to the first substrate;
wherein the microfluidic chip has a plurality of openings in communication with the primary flow channel.

In some embodiments, the primary flow channel extends in a first direction, the microfluidic channel further includes branch flow channels located on a side of the primary flow channel in a second direction, and the second direction intersects the first direction; and
at least one of the openings is in communication with the primary flow channel through at least one of the branch flow channels, respectively.

In some embodiments, the plurality of openings include a first venthole, a second venthole, a sample inlet, a binding solution inlet, a cleaning solution inlet, and an eluent inlet, and each of the sample inlet, the binding solution inlet, the cleaning solution inlet, and the eluent inlet is in communication with the primary flow channel through a corresponding one of the branch flow channels.

In some embodiments, each of the branch flow channels in communication with the sample inlet, the binding solution inlet, the cleaning solution inlet and the eluent inlet, respectively, is provided with a first valve.

In some embodiments, the branch flow channel in communication with the sample inlet, the branch flow channel in communication with the binding solution inlet, the branch flow channel in communication with the cleaning solution inlet, and the branch flow channel in communication with the eluent inlet are sequentially arranged along the first direction, the branch flow channel in communication with the binding solution inlet and the branch flow channel in communication with the sample inlet are both in communication with the primary flow channel through a first combining flow channel, and the branch flow channel in communication with the cleaning solution inlet and the branch flow channel in communication with the eluent inlet are both in communication with the primary flow channel through a second combining flow channel.

In some embodiments, the microfluidic channel further includes a connection flow channel connected between the first combining flow channel and the second combining flow channel, and the connection flow channel has a second valve disposed thereon.

In some embodiments, the first substrate further has a waste liquid tank disposed thereon, and the second venthole is in communication with the primary flow channel via the waste liquid tank.

In some embodiments, the waste liquid tank protrudes in a direction away from the second substrate.

In some embodiments, the plurality of openings further includes a waste fluid outlet in communication with the primary flow channel.

In some embodiments, the plurality of openings further includes a sample outlet in communication with the primary flow channel; and the sample outlet penetrates through the first substrate, and an end of the sample outlet distal to the second substrate is covered with a film.

In some embodiments, the primary flow channel includes a first portion and a second portion, the plurality of buffer flow channels are all located between the first portion and the second portion; and
the first venthole, the sample inlet, and the binding solution inlet are all in communication with the first portion, and the second venthole, the cleaning solution inlet, and the eluent inlet are all in communication with the second portion.

In some embodiments, the first portion of the primary flow channel has a first liquid inlet and a second liquid inlet, the second portion of the primary flow channel has a third liquid inlet and a fourth liquid inlet, the sample inlet is in communication with the first liquid inlet through a respective branch flow channel, the binding solution inlet is in communication with the second liquid inlet through a respective branch flow channel, the cleaning solution inlet is in communication with the third liquid inlet through a respective branch flow channel, and the eluent inlet is in communication with the fourth liquid inlet through a respective branch flow channel; and
the first liquid inlet is on a side of the second liquid inlet proximal to the buffer flow channels, and the third liquid inlet is on a side of the fourth liquid inlet proximal to the buffer flow channels.

In some embodiments, the microfluidic channel includes at least three buffer flow channels arranged in the first direction, the primary flow channel includes a first portion, a second portion, and connection portions, the at least three buffer flow channels are all located between the first portion and the second portion, and at least two of the buffer flow channels are in communication with each other through the connection portions, respectively;
the plurality of openings further include a waste liquid outlet and a sample outlet, the waste liquid outlet, the sample inlet, and the binding fluid inlet are all in communication with the first portion, the second venthole and the sample outlet are both in communication with the second portion, the cleaning solution inlet and the eluent inlet are in communication with the connection portions, and the cleaning solution inlet and the eluent inlet are in communication with different connection portions, respectively.

In some embodiments, the at least three buffer flow channels include a first buffer flow channel, a second buffer flow channel, a third buffer flow channel, a fourth buffer flow channel, and a fifth buffer flow channel which are arranged in sequence along the first direction, the first buffer flow channel is in communication with the first portion, the fifth buffer flow channel is in communication with the second portion, and the first buffer flow channel and the second buffer flow channel are in communication with each other, the second buffer flow channel and the third buffer flow channel are in communication with each other, and the third buffer flow channel and the fourth buffer flow channel are in communication with each other, through the connection portions, respectively; and
the microfluidic chip has two cleaning solution inlets, one of which is in communication with the connection portion between the first buffer flow channel and the second buffer flow channel through a respective branch flow channel, the other is in communication with the connection portion between the second buffer flow channel and the third buffer flow channel through a respective branch flow channel, and the eluent inlet is in communication with the connection portion between the third buffer flow channel and the fourth buffer flow channel through a respective branch flow channel.

In some embodiments, the eluent inlet and the first venthole have a one-piece structure.

In some embodiments, each of the branch flow channels has a first opening and a second opening, the first opening is in communication with the primary flow channel, the second opening is in communication with one of the inlets, at least one of the branch flow channels is a tapered flow channel, and a diameter of the first opening of the tapered flow channel is less than a diameter of the second opening of the tapered flow channel.

In some embodiments, the primary flow channel includes a first portion and a second portion, the plurality of buffer flow channels are all located between the first portion and the second portion, and the first opening of the tapered flow channel is in communication with the first portion or the second portion; and
in the first direction, a distance between the first opening of the tapered flow channel and any one of the buffer flow channels is less than a distance between the second opening of the tapered flow channel and the any one of the buffer flow channels.

In some embodiments, an orthogonal projection of each of the branch flow channels on the first substrate has a first edge and a second edge, and each of the first edge and the second edge of the tapered flow channel is arc-shaped.

In some embodiments, a width of any portion of each of the buffer flow channels is substantially the same as a width of the primary flow channel.

Embodiments of the present disclosure further provide a nucleic acid extraction apparatus, which includes the microfluidic chip according to any one of the foregoing embodiments and a magnetic control device for applying a magnetic field to the buffer areas of the microfluidic chip independently.

In some embodiments, the nucleic acid extraction apparatus further includes a mounting frame on which the microfluidic chip and the magnetic control device are both mounted, wherein
the magnetic control device includes:
a magnet mounting part;
a magnet on the magnet mounting part; and
a rotating part connected to the magnet mounting part and the mounting frame, and configured to drive the magnet mounting part to rotate around an axis of the rotating part, so as to make the magnet move between any two of an initial position and positions respectively opposite to the buffer areas, wherein the initial position and any one of the buffer areas do not overlap each other in a thickness direction of the microfluidic chip.

In some embodiments, the magnetic control device includes one magnet, the magnet mounting part is a rectangular structure and includes a first end and a second end along a lengthwise direction of the magnet mounting part, the rotating part is connected to a middle position of the magnet mounting part, and the magnet is arranged between the rotating part and the first end;
or
the magnetic control device includes a plurality of magnets, and the magnet mounting part has a shape of a circular disc, the rotating part is disposed at a center of the magnet mounting part, a peripheral portion of the magnet mounting part has a plurality of magnet installing areas and a plurality of vacant areas, each of the magnet installing areas is provided therein with one of the magnets, the plurality of magnet installing areas and the plurality of vacant areas are divided into at least one first area group, at least one second area group, and at least one third area group, each first area group includes one of the magnet installing areas and one of the vacant areas both of which are located at both ends of a diameter of the magnet mounting part, respectively, each second area group includes two of the magnet installing areas both of which are located at both ends of a diameter of the magnet mounting part, respectively, and each third area group includes two of the vacant areas both of which are located at both ends of a diameter of the magnet mounting part, respectively.

In some embodiments, the nucleic acid extraction apparatus further includes a mounting frame on which the microfluidic chip and the magnetic control device are both mounted, wherein
the magnetic control device includes:
a guide rail extending in a direction in which the buffer areas of the microfluidic chip are arranged;
a magnet mounting part on the guide rail; and
a magnet on the magnet mounting part;
wherein the magnet mounting part is configured to move along the guide rail to make the magnet move between any two of an initial position and positions respectively opposite to the buffer areas, and the initial position and any one of the buffer areas do not overlap each other in a thickness direction of the microfluidic chip.

The present disclosure further provides a nucleic acid extraction method for the microfluidic chip according to any one of the foregoing embodiments, the nucleic acid extraction method including:
introducing a mixed solution of a sample solution, a sample lysis solution and magnetic beads into the primary flow channel through the openings, to allow a sample in the sample solution to release a nucleic acid under an action of the sample lysis solution, and the nucleic acid to be attached to the magnetic beads;
applying a magnetic field to one of the buffer areas to adsorb the magnetic beads to the one of the buffer areas;
discharging a waste liquid from the microfluidic channel;
withdrawing the magnetic field in the buffer area where the magnetic beads are located currently, applying a magnetic field to one of the remaining buffer areas, and introducing a binding solution into the primary flow channel through the openings, to allow the magnetic beads to be resuspended by the binding solution and then to be adsorbed in the buffer area where the magnetic field exists;
discharging a waste liquid from the microfluidic channel;
withdrawing the magnetic field in the buffer area where the magnetic beads are located currently, and introducing a cleaning solution into the primary flow channel through the openings, to allow the magnetic beads to be resuspended and cleaned by the cleaning solution;
applying a magnetic field to at least one of the buffer areas to adsorb the magnetic beads, and discharging a waste liquid from the microfluidic channel;
introducing an eluent into the primary flow channel through the openings, and withdrawing the magnetic field in the buffer area where the magnetic beads are located currently, to allow the magnetic beads to be resuspended by the eluent, and the nucleic acid to be separated from the magnetic beads;
applying a magnetic field to at least one of the buffer areas to adsorb the magnetic beads; and
discharging a solution mixed with the nucleic acid from the microfluidic channel.

In some embodiments, the mixed solution is introduced into the primary flow channel through the sample inlet, the binding solution is introduced into the primary flow channel through the binding solution inlet, the cleaning solution is introduced into the primary flow channel through the cleaning solution inlet, and the eluent is introduced into the primary flow channel through the eluent inlet; and
the waste liquid is discharged each time by sucking air from or introducing air into one of the first venthole and the second venthole.

In some embodiments, the discharging the solution mixed with the nucleic acid from the microfluidic channel includes:
breaking the film mechanically, and sucking air from or introducing air into one of the first venthole and the second venthole, to allow the solution mixed with the nucleic acid to be discharged from the microfluidic chip through the sample outlet.

In some embodiments, prior to the introducing the eluent into the primary flow channel through the openings, the nucleic acid extraction method further includes:
sucking air from or introducing air into one of the first venthole and the second venthole, to remove the waste liquid remaining in the microfluidic channel.

### BRIEF DESCRIPTION OF THE DRAWINGS

The accompanying drawings, which are included to provide a further understanding of the present disclosure and constitute a part of this specification, explain the present disclosure together with the following exemplary embodiments, but are not intended to limit the present disclosure. In the drawings:

FIG. 1A is a perspective view of a microfluidic chip according to some embodiments of the present disclosure.

FIG. 1B is a top view of a microfluidic chip according to some embodiments of the present disclosure.

FIG. 2 is a schematic diagram showing two kinds of buffer flow channels in a comparative example.

FIG. 3 is a top view of a microfluidic chip according to other embodiments of the present disclosure.

FIG. 4A is a top view of a microfluidic chip according to further embodiments of the present disclosure.

FIG. 4B is a cross-sectional view taken along a line A-A′ as shown in FIG. 4A.

FIGS. 4C to 4E are various schematic diagrams each showing a distribution of a first portion of a primary flow channel and branch flow channels connected to the first portion, according to some embodiments of the present disclosure.

FIG. 5 is a top view of a microfluidic chip according to further embodiments of the present disclosure.

FIG. 6 is a top view of a microfluidic chip according to further embodiments of the present disclosure.

FIG. 7A is a top view of a microfluidic chip according to further embodiments of the present disclosure.

FIG. 7B is a schematic diagram showing a connection between one of the branch flow channels and the primary flow channel shown in FIG. 7A.

FIG. 8 is a top view of a magnetic control device according to some embodiments of the present disclosure.

FIG. 9 is a top view of a magnetic control structure according to other embodiments of the present disclosure.

FIG. 10 is a top view of a magnetic control structure according to further embodiments of the present disclosure.

FIG. 11 is a schematic diagram of a nucleic acid extraction method according to some embodiments of the present disclosure.

### DETAILED DESCRIPTION OF EMBODIMENTS

To make the objects, technical solutions and advantages of the embodiments of the present disclosure more apparent, the technical solutions of the embodiments of the present disclosure will be clearly and completely described below with reference to the accompanying drawings the embodiments of the present disclosure. It is to be understood that the described embodiments are only a few, but not all, embodiments of the present disclosure. All other embodiments, which may be derived by one of ordinary in the art from the described embodiments of the present disclosure without an inventive effort, fall within the scope of protection of the present disclosure.

Unless defined otherwise, technical or scientific terms used herein shall have the ordinary meaning as understood by one of ordinary skill in the art to which the present disclosure belongs. The terms “first”, “second”, and the like used in the present disclosure are not intended to indicate any order, quantity, or importance, but rather are used for distinguishing one element from another. Further, the terms “a”, “an”, “the”, or the like does not denote a limitation of quantity, but rather denotes the presence of at least one element. The term of “comprising”, “including”, or the like means that the element or item preceding the term contains the element or item listed after the term and its equivalent, but does not exclude the presence of other elements or items. The term “connected”, “coupled”, or the like is not limited to physical or mechanical connections, but may include electrical connections, whether direct or indirect connections. The terms “upper”, “lower”, “left”, “right”, and the like are used only for indicating relative positional relationships, and when the absolute position of an object being described is changed, the relative positional relationships may also be changed accordingly.

A microfluidic chip is widely used in the field of biochemical analysis, such as the field of nucleic acid detection, due to its characteristics of easy integration, automation, controllable fluid and small required sample volume. In some embodiments, the microfluidic chip may be used for nucleic acid extraction (i.e., for extracting nucleic acid(s)), and the nucleic acid extraction includes: sequentially introducing a plurality of reagents into a flow channel in the microfluidic chip, where the plurality of reagents include, for example, a sample mixed solution, a binding solution, a cleaning solution, and an eluent, and the sample mixed solution may include a sample solution, a sample lysis solution, and magnetic beads. The sample solution can release nucleic acids under the lysis effect of the sample lysis solution, and the nucleic acids are attached to the magnetic beads. After the sample mixed solution is introduced into the flow channel, the magnetic beads having the nucleic acids attached thereto are adsorbed in a region where a magnetic field is applied, and the resultant waste liquid is discharged from the flow channel. When the binding solution is introduced into the flow channel, the magnetic field in the region where the magnetic beads are currently located is removed, such that the magnetic beads are resuspended by the binding solution. The nucleic acids are more stably attached to the magnetic beads by the action of the binding solution, and after the binding solution is sufficiently in contact with the nucleic acids, a magnetic field is applied to fix the magnetic beads and discharge the resultant waste liquid from the flow channel. When the cleaning solution is introduced into the flow channel, the magnetic field in the current region where the magnetic beads are located is removed, such that the magnetic beads are resuspended by the cleaning solution. After the magnetic beads are sufficiently in contact with the cleaning solution, a magnetic field is applied to fix the magnetic beads and discharge the resultant waste liquid out of the flow channel. When the eluent is introduced into the flow channel, the magnetic field in the current region where the magnetic beads are located is removed, such that the magnetic beads are resuspended by the eluent, and the magnetic beads are sufficiently in contact with the eluent, thereby separating the nucleic acids from the magnetic beads under the action of the eluent; and a magnetic field is applied to fix the magnetic beads and discharge the resultant waste liquid out of the flow channel.

In some microfluidic chips, because the magnetic beads have a large density and a large weight, an efficiency of the resuspension of the magnetic beads is low after a reagent is introduced into the flow channel of each of the microfluidic chips, resulting in insufficient contact between the magnetic beads and the reagent, and thus a reduced magnetic bead extraction efficiency.

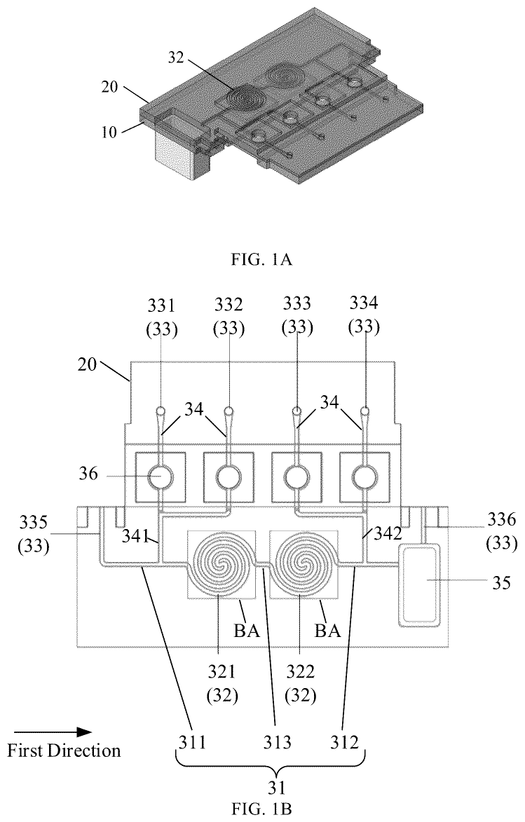

In order to solve the above problems, embodiments of the present disclosure provide a microfluidic chip for performing nucleic acid extraction (i.e., for extracting nucleic acid(s)). FIG. 1A is a perspective view of a microfluidic chip according to some embodiments of the present disclosure, and FIG. 1B is a top view of a microfluidic chip according to some embodiments of the present disclosure. As shown in FIGS. 1A and 1B, the microfluidic chip includes a first substrate 10 and a second substrate 20 disposed opposite to each other. Each of the first substrate 10 and the second substrate 20 may be a glass substrate, and alternatively, may be another substrate made of a suitable material, which is not limited in an embodiment of the present disclosure. Each of the first substrate 10 and the second substrate 20 may have a rectangular shape, or another suitable shape, which is not limited in an embodiment of the present disclosure. Further, the second substrate 20 may have the same shape and the same size as those of the first substrate 10.

The first substrate 10 has at least two buffer areas BA for cooperating with a magnet, and a magnetic field may be applied to the buffer areas BA when the microfluidic chip performs nucleic acid extraction. It should be noted that not all of the buffer areas BA are simultaneously applied with a magnetic field, and for example, at most one of the buffer areas BA is in a magnetic field at any time. The first substrate 10 is provided thereon with a microfluidic channel, and the microfluidic channel includes a primary flow channel (or a main flow channel) 31 and a plurality of buffer flow channels 32 connected to the primary flow channel 31 in series. The buffer flow channels 32 are disposed in the buffer areas BA, and different buffer flow channels 32 are disposed in different buffer areas BA, respectively. Each of the buffer flow channels 32 has a double-spiral shape. The microfluidic chip has a plurality of openings 33, and the plurality of openings 33 each communicate with the primary flow channel 31.

In the present embodiment, each of the buffer flow channels 32 is configured to have the double-spiral shape, and thus has an increased length under the condition that a corresponding buffer area BA has a fixed area. As such, a path through which a reagent flows in each buffer flow channel 32 is relatively long, thereby prolonging the time for which the reagent and the magnetic beads interact with each other, which is beneficial to improving the efficiency of the resuspension of the magnetic beads, and further improving the nucleic acid extraction efficiency.

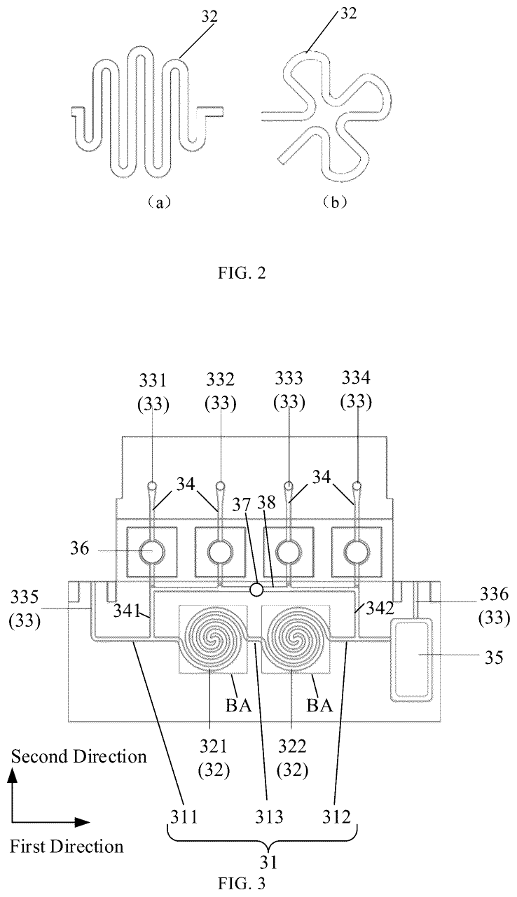

FIG. 2 is a schematic diagram showing two kinds of buffer flow channels in a comparative example. As shown in FIG. 2, the buffer flow channel 32 shown in the figure (a) has a serpentine structure, and the buffer flow channel 32 shown in the figure (b) has a petal-shaped structure. Taking a circular area with a radius of 8 mm as an example, in the cases where the circular area is respectively provided with the buffer flow channel 32 having the double-spiral shape, the buffer flow channel 32 having the serpentine structure and the buffer flow channel 32 having petal-shaped structure, a length of the buffer flow channel 32 having the double-spiral shape can reach about 116.42 mm, a length of the buffer flow channel 32 having the serpentine structure can reach about 89 mm, and a length of the buffer flow channel 32 having petal-shaped structure can reach 64.47 mm. In addition, compared with the two buffer flow channels shown in FIG. 2, in the buffer flow channel 32 having the double-spiral shape according to an embodiment of the present disclosure, a liquid flows along the channel with gradually changing curvature and thus is subjected to a small interference, and a change of a flow speed of the liquid is largest at a central position having the largest curvature, but a density of a magnetic field is largest at the central position, such that the magnetic beads are not easily dispersed by the flow of the liquid, and the overall capture efficiency is relatively uniform. For each buffer flow channel 32, the flow speed of the liquid changes greatly in an arc region of the flow channel, and the magnetic field at this region is weaker than the magnetic field at the central position. As such, the magnetic beads are easily captured in a vertical portion of the flow channel. An amount of liquid that can be accommodated in the buffer flow channel 32 is small, and thus a utilization rate of the magnetic field is low.

It should be understood that if a width of each buffer flow channel 32 is too large, the flow speed of a reagent flowing into the buffer flow channel 32 after flowing through the primary flow channel 31 may be rapidly reduced, such that the magnetic beads are prone to sedimentation, which affects the resuspension efficiency of the magnetic beads. To avoid this phenomenon, in some embodiments, a width of any portion of each buffer flow channel 32 is substantially the same as a width of the primary flow channel 31. The expression “substantially the same” means that two values differ by a certain amount, which is, for example, less than 5%, or 10%, or 15%. It should be further noted that the primary flow channel 31 may extend along a first direction, and the width of the primary flow channel 31 is a dimension (i.e., size) of the primary flow channel 31 in a direction perpendicular to an extending direction of the primary flow channel 31. The width of any portion of each buffer flow channel 32 is a dimension (i.e., size) of the any portion in a direction perpendicular to the tangent of the any portion.

In some embodiments, as shown in FIG. 1B, the microfluidic channel of the microfluidic chip further includes branch flow channels 34 located on a side of the primary flow channel 31 in a second direction intersecting the first direction, and for example, the first direction is perpendicular to the second direction. At least one of the openings 33 is in communication with the primary flow channel 31 through corresponding branch flow channel(s) 34.

In some embodiments, as shown in FIG. 1B, the plurality of openings 33 include a plurality of ventholes and a plurality of reagent inlets. The plurality of ventholes include, for example, a first venthole 335 and a second venthole 336, and the plurality of reagent inlets include, for example, a sample inlet 331, a binding solution inlet 332, a cleaning solution inlet 333, and an eluent inlet 334. The sample inlet 331 may be in communication with an external first liquid storage chamber, so as to introduce a solution provided from the first liquid storage chamber into the microfluidic channel, where the solution is a mixed solution of a sample solution, a sample lysis solution, and magnetic beads. Apparently, in a case where the sample lysis solution is already present in the microfluidic channel, the solution provided from the first liquid storage chamber may be a mixed solution of the sample solution and the magnetic beads. The binding solution inlet 332 may be in communication with an external sample injector for introducing the binding solution provided from the sample injector into the microfluidic channel. The cleaning solution inlet 333 may be in communication with an external second liquid storage chamber for introducing the cleaning solution provided from the second liquid storage chamber into the microfluidic channel. The eluent inlet 334 may be in communication with a third liquid storage chamber for introducing the eluent provided from the third liquid storage chamber into the microfluidic channel. The first venthole 335 and the second venthole 336 are connected to an air pump for ventilating or evacuating the microfluidic channel, so as to push the reagent in the microfluidic channel to flow and mix the reagent in each buffer flow channel 32 with the magnetic beads.

In an example, as shown in FIG. 1B, the first venthole 335 and the second venthole 336 are located on the first substrate 10, and for example, the first venthole 335 and the second venthole 336 may extend from the primary flow channel 31 to a side of first substrate 10 in a direction parallel to the first substrate 10. For another example, the first venthole 335 and the second venthole 336 may be provided on the second substrate 20, and penetrate through the second substrate 20 in a thickness direction of the second substrate 20.

A shape of each of the openings 33 is not limited in an embodiment of the present disclosure, and for example, in the case where each opening 33 penetrates through the second substrate 20 in the thickness direction of the second substrate 20, a shape of an orthogonal projection of each opening 33 on the first substrate 10 may be a circle, a rectangle, an irregular shape, or the like.

In some embodiments, as shown in FIG. 1B, each of the sample inlet 331, the binding solution inlet 332, the cleaning solution inlet 333, and the eluent inlet 334 is in communication with the primary flow channel 31 through a corresponding branch flow channel 34. Each branch flow channel 34 may have a straight-line shape, and an extending direction of each branch flow channel 34 may intersect the extending direction of the primary flow channel 31. For example, the extending direction of each branch flow channel 34 is substantially perpendicular to the extending direction of the primary flow channel 31, and alternatively, each branch flow channel 34 may have a curved shape.

In an example, the branch flow channel 34 connected to the sample inlet 331, the branch flow channel 34 connected to the binding solution inlet 332, the branch flow channel 34 connected to the cleaning solution inlet 333, and the branch flow channel 34 connected to the eluent inlet 334 are sequentially arranged along the first direction.

In some embodiments, each of the branch flow channel 34 connected to the sample inlet, the branch flow channel 34 connected to the binding solution inlet 332, the branch flow channel 34 connected to the cleaning solution inlet 333, and the branch flow channel 34 connected to the eluent inlet 334 is provided thereon with a first valve 36, for controlling the on/off of the branch flow channel. When a reagent needs to be introduced into the inlet corresponding to the reagent, the corresponding first valve 36 is turned on. The first valve 36 may be a magnetic valve.

Optionally, the microfluidic channel further includes a first combining flow channel 341 and a second combining flow channel 342. The branch flow channel 34 connected to the sample inlet and the branch flow channel 34 connected to the binding solution inlet 332 are both connected to a liquid inlet of the first combining flow channel 341, and a liquid outlet of the first combining flow channel 341 is in communication with the primary flow channel 31. The branch flow channel 34 in communication with the cleaning solution inlet 333 and the branch flow channel 34 in communication with the eluent inlet 334 are both in communication with a liquid inlet of the second combining flow channel 342, and a liquid outlet of the second combining flow channel 342 is in communication with the primary flow channel 31.

For example, as shown in FIG. 1B, the branch flow channel 34 in communication with the sample inlet 331 and the first combining flow channel 341 both have straight-line shapes, respectively, and the branch flow channel 34 in communication with the binding solution inlet 332 has a curved shape. The branch flow channel 34 in communication with the eluent inlet 334 and the second combining flow channel 342 both have straight-line shapes, respectively, and the branch flow channel 34 in communication with the cleaning solution inlet 333 has a curved shape. Alternatively, the branch flow channel 34 in communication with the sample inlet 331 and the branch flow channel 34 in communication with the eluent inlet 334 may have curved shapes, respectively, the branch flow channel 34 in communication with the binding solution inlet 332 and the first combining flow channel 341 may have straight-line shapes, respectively, and the branch flow channel 34 in communication with the cleaning solution inlet 333 and the second combining flow channel 342 may have straight-line shapes, respectively.

In some embodiments, as shown in FIG. 1B, the primary flow channel 31 includes a first portion 311, a second portion 312, and a connection portion 313. The plurality of buffer flow channels 32 are all located between the first portion 311 and the second portion 312, and the connection portion 313 is located between any adjacent two of the buffer flow channels 32. Each branch flow channel 34 may be in communication with the first portion 311 or the second portion 312. For example, in FIG. 1B, the microfluidic channel may include two buffer flow channels 32, the branch flow channel 34 in communication with the sample inlet 331 and the branch flow channel 34 in communication with the binding solution inlet 332 are in communication with the first portion 311 through the first combining flow channel 341, and the branch flow channel 34 in communication with the cleaning solution inlet 333 and the branch flow channel 34 in communication with the eluent inlet 334 are in communication with the second portion 312 through the second combining flow channel 342. The first venthole 335 and the second venthole 336 are respectively in communication with both ends of the primary flow channel 31, and the reagent and the magnetic beads are controlled to flow between the plurality of buffer flow channels 32 by performing air suction (air-suction operation) or air ventilation (air-supply operation) on one of the first venthole 335 and the second venthole 336 (i.e., sucking air from or introducing air into one of the first venthole 335 and the second venthole 336). For example, the first venthole 335 is in communication with the first portion 311 of the primary flow channel 31, and the second venthole 336 is in communication with the second portion 312 of the primary flow channel 31.

In an example, the first venthole 335 and the second venthole 336 are both located on first substrate 10. For example, one end of the first venthole 335 is in direct communication with the first portion 311 of the primary flow channel 31, and the other end of the first venthole 335 extends to a side of the first substrate 10. In some embodiments, a waste liquid tank 35 is further disposed on the first substrate 10, and the second venthole 336 is in communication with the primary flow channel 31 via the waste liquid tank 35. For example, one end of the second portion 312 of the primary flow channel 31 is in communication with a buffer flow channel 32, and the other end of the second portion 312 is in communication with the waste liquid tank 35. One end of the second venthole 336 is in communication with the waste liquid tank 35, and the other end of the second venthole 336 extends to an end surface of the first substrate 10. After the reagent and the magnetic beads are sufficiently contacted to each other, the remaining waste solution (or liquid) can be introduced into the waste liquid tank 35 by performing an air-supply or air-suction operation on the first venthole 335 or the second venthole 336 (i.e., sucking air from or introducing air into the first venthole 335 or the second venthole 336).

The waste liquid tank 35 may protrude away from the second substrate 20, such that a waste liquid may, under the action of gravity, flow to the bottom of the waste liquid tank 35 after getting into the waste liquid tank 35, thereby preventing the waste liquid from flowing back into the primary flow channel 31, and improving a storage effect of the waste liquid tank. In addition, a water absorbing material may be further disposed in the waste liquid tank 35, so as to further improve the storage effect of the waste liquid tank 35.

A process of extracting nucleic acid(s) (i.e., a nucleic acid extraction process) using the microfluidic chip shown in FIGS. 1A and 1B, in which the two buffer flow channels 32 are respectively referred to as a first buffer flow channel 321 and a second buffer flow channel 322, will be described below. The nucleic acid extraction process may include the following steps S11 to S15.

Step S11 includes applying a magnetic field to the buffer area where the first buffer flow channel 321 is located, but not applying a magnetic field to the buffer area where the second buffer flow channel 322 is located; introducing the mixed solution of the sample solution, the sample lysis solution and the magnetic beads into the microfluidic channel through the sample inlet 331, turning on the first valve 36 on the branch flow channel 34 in communication with the sample inlet 331, and turning off the remaining valves. Further, the first venthole 335 is closed, and a negative pressure is applied to the second venthole 336, such that the mixed solution flows into the first buffer flow channel 321, the magnetic beads are adsorbed in the buffer area where the first buffer flow channel 321 is located, nucleic acid(s) in the sample solution is (are) attached to the magnetic beads, and the rest of the solution enters the waste liquid tank 35 as a waste solution (or a waste liquid).

Step S12 includes introducing a binding solution into the microfluidic channel through the binding solution inlet 332, turning on the first valve 36 on the branch flow channel 34 in communication with the binding solution inlet 332, and turning off the remaining first valves 36; withdrawing the magnetic field in the buffer area where the first buffer flow channel 321 is located, and applying a magnetic field to the buffer area where the second buffer flow channel 322 is located; and applying a negative pressure to the second venthole 336, such that the binding solution flows into the second buffer flow channel 322 through the first buffer flow channel 321, the magnetic beads are resuspended by the binding solution in this process and then are adsorbed in the second buffer flow channel 322, and the waste solution (or liquid) without interaction with the magnetic beads enters the waste liquid tank 35.

Step S13 includes introducing a cleaning solution into the microfluidic channel through the cleaning solution inlet 333, turning on the first valve 36 on the branch flow channel 34 in communication with the cleaning solution inlet 333, and turning off the rest first valves 36; withdrawing the magnetic field in the buffer area where the second buffer flow channel 322 is located, and applying a magnetic field to the buffer area where the first buffer flow channel 321 is located; and applying a negative pressure to the first venthole 335, such that the cleaning solution enters the first buffer flow channel 321 through the second buffer flow channel 322, and the cleaning solution is absorbed in the first buffer flow channel 321 after flowing through the second buffer flow channel 322. At this time, the first valve 36 on the branch flow channel 34 in communication with the cleaning solution inlet 333 is turned off. When a liquid block flows from the first buffer flow channel 321, the first venthole is closed, a negative pressure is applied to the second venthole 336, the magnetic field in the buffer area where the first buffer flow channel 321 is located is withdrawn, and a magnetic field is applied to the buffer area where the second buffer flow channel 322 is located, such that the magnetic beads are resuspended by the cleaning solution again, and this process may be repeated for multiple times to improve the cleaning effect. Finally, a magnetic field is applied to the buffer area where the second buffer flow channel 322 is located, the magnetic field in the buffer area where the first buffer flow channel 321 is located is withdrawn, and a negative pressure is applied to the second venthole 336, such that the waste liquid (or solution) generated upon the completion of the cleaning process enters the waste liquid tank 35.

Here, step S13 may be performed once or repeatedly for a plurality of times.

Step S14 includes keeping the applying of the magnetic field to the buffer area where the second buffer flow channel 322 is located, keeping the negative pressure at the second venthole 336, and opening the first venthole 335, thereby sucking the residual liquid in the microfluidic channel into the waste liquid tank 35.

Step S15 includes introducing an eluent into the microfluidic channel through the eluent inlet 334, turning on the first valve 36 on the branch flow channel 34 in communication with the eluent inlet 334, and turning off the remaining first valves 36; withdrawing the magnetic field in the buffer area where the second buffer flow channel 322 is located, applying a magnetic field to the buffer area where the first buffer flow channel 321 is located, and applying a negative pressure to the first venthole 335, such that the eluent flows into the first buffer flow channel 321 through the second buffer flow channel 322, and in this process, the magnetic beads are resuspended by the eluent and then adsorbed in the first buffer flow channel 321. After the introduction of the eluent is completed, the first valve 36 on the branch flow channel 34 in communication with the eluent inlet 334 is turned off. When a liquid is to flow out of the first buffer flow channel 321, the first venthole 335 is closed, a negative pressure is applied to the second venthole 336, the magnetic field in the buffer area where the first buffer flow channel 321 is located is withdrawn, and a magnetic field is applied to the buffer area where the second buffer flow channel 322 is located, such that magnetic beads in the first buffer flow channel 321 are resuspended, then the eluent flows into the second buffer flow channel 322 and is adsorbed in the second buffer flow channel 322, and this process may be repeated for multiple times such that the eluent is in sufficient contact with the magnetic beads, and the elution effect is improved. Finally, a magnetic field is applied to the buffer area where at least one of the first buffer flow channel 321 and the second buffer flow channel 322 is located (for example, a magnetic field may be applied to both the buffer area where the first buffer flow channel 321 is located and the buffer area where the second buffer flow channel 322 is located at the same time to improve the adsorption effect on the magnetic beads and thus the nucleic acid extraction efficiency), and a negative pressure is applied to the first venthole 335, such that the solution mixed with nucleic acid(s) is discharged from the first venthole 335, thereby completing the nucleic acid extraction.

It should be understood that after the eluent is in sufficient contact with the magnetic beads, nucleic acid(s) attached to the magnetic beads are separated from the magnetic beads under the action of the eluent, and at this time, the solution in the microfluidic channel is the solution mixed with the nucleic acid(s).

FIG. 3 is a top view of a microfluidic chip according to other embodiments of the present disclosure. The microfluidic chip shown in FIG. 3 is similar to that shown in FIG. 1B except that the microfluidic chip shown in FIG. 3 further includes a connection flow channel connected between the first combining flow channel 341 and the second combining flow channel 342, and a second valve 38 arranged on the connection flow channel, the second valve 38 being used for controlling the on-off of the connection flow channel. When the second valve 38 is turned on, the first venthole 335 is opened, and a negative pressure is applied to the second venthole 336, a flow resistance on a path where the first combining flow channel 341, the second combining flow channel 342 and the connection flow channel are located is smaller than a flow resistance on a path where the first buffer flow channel 321 and the second buffer flow channel 322 are located, such that an airflow preferentially passes through the path where the first combining flow channel 341, the second combining flow channel 342 and the connection flow channel are located, thereby allowing the residual liquid in the first combining flow channel 341 and the second combining flow channel 342 to be sucked into the waste liquid tank 35, preventing the residual liquid from affecting a subsequent introduced reagent, and thus improving the nucleic acid extraction efficiency.

A process of extracting nucleic acid(s) by using the microfluidic chip shown in FIG. 3 is similar to the steps S11 to S15, except that the process of extracting nucleic acid(s) by using the microfluidic chip shown in FIG. 3 further includes a step S135 between the steps S13 and S14, and step S135 includes: keeping the applying of a magnetic field to the buffer area where the second buffer flow channel 322 is located, turning on the second valve 38, keeping the negative pressure at the second venthole 336, and opening the first venthole 335, such that the residual liquid in the first combining flow channel 341 and the second combining flow channel 342 is sucked into the waste liquid tank 35. Further, for the microfluidic chip shown in FIG. 3, the first valves 36 are turned off in each of steps S11, S12, S13, S14, and S15.

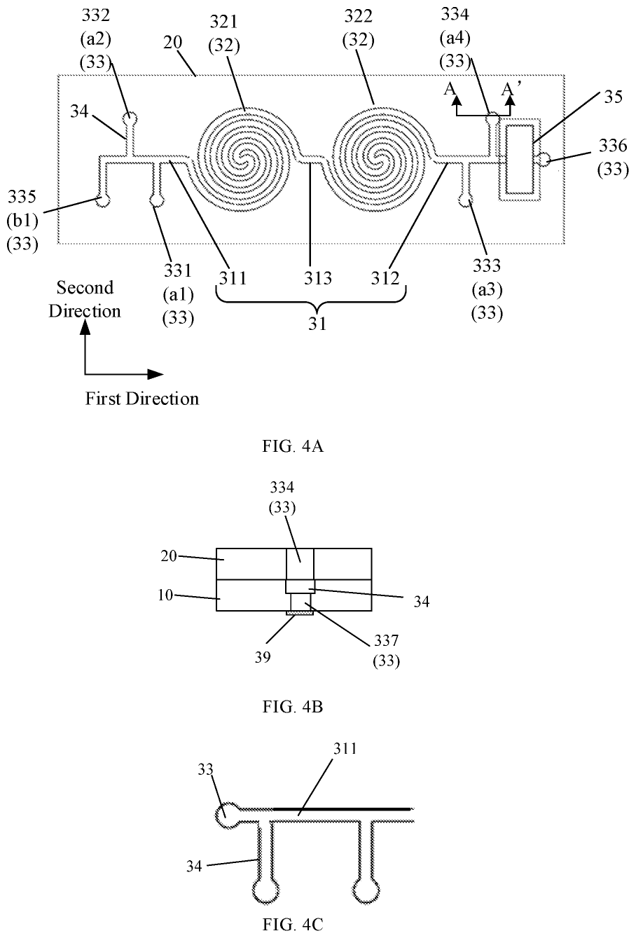

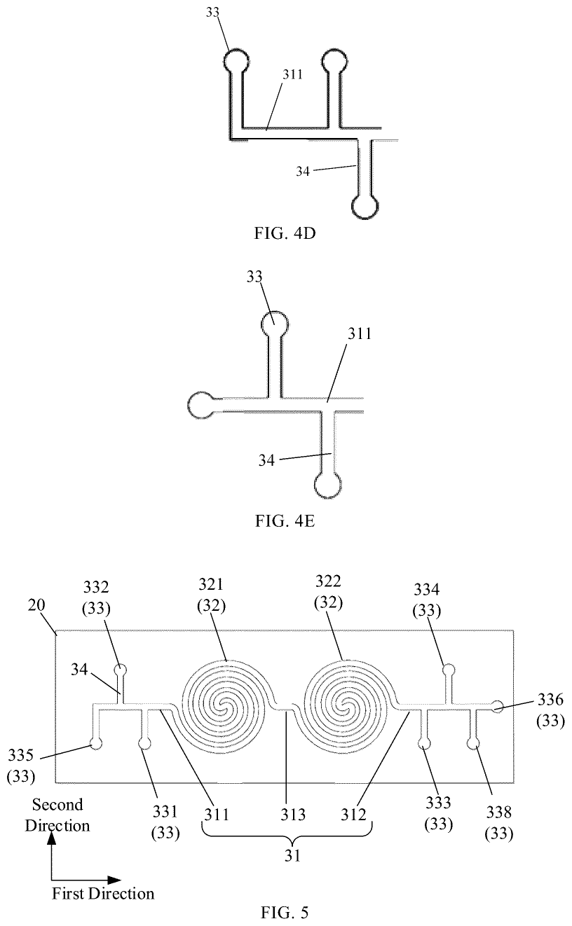

FIG. 4A is a top view of a microfluidic chip according to further embodiments of the present disclosure, and FIG. 4B is a cross-sectional view taken along a line A-A′ as shown in FIG. 4A. The microfluidic chip shown in FIG. 4A includes a first substrate 10 and a second substrate 20 disposed opposite to each other, similar to the microfluidic chip shown in FIG. 1B. The first substrate 10 has at least two buffer areas BA for cooperating with a magnet 42 (which is shown in each of FIGS. 8 to 10). A microfluidic channel is provided on the first substrate 10, and includes a primary flow channel 31 and a plurality of buffer flow channels 32 connected in series to the primary flow channel 31. The buffer flow channels 32 are provided in the buffer areas BA, and different buffer flow channels 32 are provided in different buffer areas BA, respectively. Each buffer flow channel 32 has a double-spiral shape. The microfluidic chip has a plurality of openings 33 in communication with the primary flow channel 31.

As in FIG. 1B, in FIG. 4A, a width of any portion of each buffer flow channel 32 is substantially the same as a width of the primary flow channel 31.

Similar to FIG. 1B, the primary flow channel 31 shown in FIG. 4A includes a first portion 311, a second portion 312, and a connection portion 313, the plurality of buffer flow channels 32 are each located between the first portion 311 and the second portion 312, and the connection portion 313 is located between any adjacent two of the buffer flow channels 32. The first substrate 10 is provided thereon with a waste liquid tank 35, and the waste liquid tank 35 protrudes in a direction away from the second substrate 20. In an example, the plurality of buffer flow channels 32 include two buffer flow channels 32.

Optionally, in FIG. 4A, the microfluidic channel of the microfluidic chip further includes branch flow channels 34 respectively located on sides of the primary flow channel 31 in a second direction intersecting the first direction, and for example, the first direction is perpendicular to the second direction. The plurality of openings 33 includes a first venthole 335, a second venthole 336, a sample inlet 331, a binding solution inlet 332, a cleaning solution inlet 333, and an eluent inlet 334. The sample inlet 331, the binding solution inlet 332, the cleaning solution inlet 333 and the eluent inlet 334 are all in communication with the primary flow channel 31 through the respective branch flow channels 34. Each branch flow channel 34 may have a straight-line shape, and an extending direction of each branch flow channel 34 may intersect an extending direction of the primary flow channel 31, and for example, the extending direction of each branch flow channel 34 is substantially perpendicular to the extending direction of the primary flow channel 31. Here, each branch flow channel 34 may be in communication with the first portion 311 or the second portion 312.

Unlike FIG. 1B, in FIG. 4A, both the first venthole 335 and the second venthole 336 may be disposed on the second substrate 20. In addition, unlike FIG. 1B, in FIG. 4A, no first valve 36 may be provided, and the plurality of branch flow channels 34 are not disposed on a same side of the primary flow channel 31, but are distributed on both sides of the primary flow channel 31.

For example, the branch flow channel 34 in communication with the binding solution inlet 332 and the branch flow channel 34 in communication with the eluent inlet 334 are both located on an upper side (i.e., the upper side in FIG. 4A) of the primary flow channel 31, and the branch flow channel 34 in communication with the first venthole 335, the branch flow channel 34 in communication with the sample inlet 331, and the branch flow channel 34 in communication with the cleaning solution inlet 333 are all located on a lower side (i.e., the lower side in FIG. 4A) of the primary flow channel 31.

Alternatively, the branch flow channels 34 may be arranged in other manners, and FIGS. 4C to 4E are various schematic diagrams each showing a distribution of the first portion 311 of the primary flow channel 31 and the branch flow channels 34 connected to the first portion 311, according to some embodiments of the present disclosure. As shown in FIGS. 4C to 4E, in the case where the first portion 311 of the primary flow channel 31 is connected to multiple branch flow channels 34, for example, as shown in FIG. 4C, the multiple branch flow channels 34 may be located on a same side of the first portion 311 of the primary flow channel 31, and an orthogonal projection of one of the openings 33 in communication with the first portion 311 on the first substrate 10 may be located on a straight line where the primary flow channel 31 is located. For another example, as shown in FIG. 4D, some of the branch flow channels 34 connected to the first portion 311 are located on one side of the first portion 311, and the others of the branch flow channels 34 connected to the first portion 311 are located on the other side of the first portion 311. For another example, as shown in FIG. 4E, an orthogonal projection of one of the openings 33 in communication with the first portion 311 on the first substrate 10 may be located on the straight line where the primary flow channel 31 is located, and the plurality of branch flow channels 34 are respectively located on both sides of the first portion 311 of the primary flow channel 31. Similarly, multiple branch flow channels 34 connected to the second portion 312 of the primary flow channel 31 may also be distributed in various manners, which is not limited in the present disclosure.

As shown in FIG. 4A, the first portion 311 of the primary flow channel 31 has a first liquid inlet a1, a first gas inlet b1, and a second liquid inlet a2, where the branch flow channel 34 in communication with the bonding solution inlet 332 is in communication with the second liquid inlet a2, the branch flow channel 34 in communication with the first venthole 335 is in communication with the first gas inlet b1, and the branch flow channel 34 in communication with the sample inlet 331 is in communication with the first liquid inlet a1. Optionally, the second liquid inlet a2 is closer to a buffer flow channel 32 than the first gas inlet b1, and the first liquid inlet a1 is closer to the buffer flow channel 32 than the second liquid inlet a2. In this case, when a bonding solution is introduced into the primary flow channel 31 through the bonding solution inlet 332, the solution remained in the primary flow channel 31 previously can be brought into the buffer flow channel 32, and thus an influence on a subsequent reaction process can be reduced.

The second portion 312 of the primary flow channel 31 has a third liquid inlet a3, a fourth liquid inlet a4, and a second gas inlet (not shown), where the branch flow channel 34 in communication with the cleaning solution inlet 333 is in communication with the third liquid inlet a3, the branch flow channel 34 in communication with the eluent inlet 334 is in communication with the fourth liquid inlet a4, an orthogonal projection of the second venthole 336 on the first substrate 10 is located on an extension line of the primary flow channel 31, and the second venthole 336 may be in communication with the second gas inlet through the waste liquid tank 35. Here, the third liquid inlet a3 is closer to a buffer flow channel 32 than the fourth liquid inlet a4. When an eluent is introduced into the primary flow channel 31 through the eluent inlet 334, the cleaning solution remained in the primary flow channel 31 previously can be brought into the buffer flow channel 32, thereby reducing an influence on a subsequent reaction process.

It should be noted that the first portion 311 and the second portion 312 of the primary flow channel 31 may be in communication with a greater number of branch flow channels 34, and in this case, the branch flow channels 34 may be disposed according to the above design principle. That is, a branch flow channel 34 corresponding to a reagent that is first introduced into the microfluidic channel may be in communication with the primary flow channel 31 at a position closer to a buffer flow channel 32.

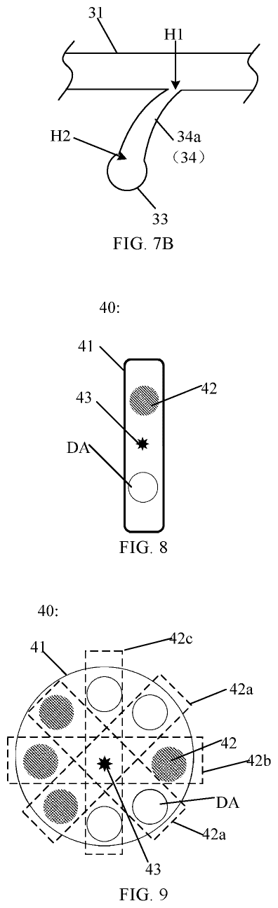

Unlike FIG. 1B, in the microfluidic chip shown in FIG. 4A, the plurality of openings 33 further includes a sample outlet 337 (which is shown in FIG. 4B or 7A) in communication with the primary flow channel 31. In some embodiments, the sample outlet 337 penetrates through the first substrate 10, and an end of the sample outlet 337 distal to the second substrate 20 is covered with a film 39, which is mechanically blasted when it is desired to allow a sample solution to flow out of the microfluidic chip. Exemplarily, the eluent inlet 334 penetrates through the second substrate 20, and the sample outlet 337 may be disposed opposite to the eluent inlet 334.

A process of extracting nucleic acid(s) (i.e., a nucleic acid extraction process) by using the microfluidic chip shown in FIG. 4A will be described below, in which the two buffer flow channels 32 are respectively referred to as a first buffer flow channel 321 and a second buffer flow channel 322. The nucleic acid extraction process includes the following steps S21 to S26.

Step S21 includes applying a magnetic field to the buffer area where the first buffer flow channel 321 is located, but not applying a magnetic field to the buffer area where the second buffer flow channel 322 is located; introducing a mixed solution of a sample solution, a sample lysis solution and magnetic beads into the microfluidic channel through the sample inlet 331, opening the second venthole 336, and introducing air into the first venthole 335, such that the mixed solution flows into the first buffer flow channel 321, the magnetic beads are adsorbed in the buffer area where the first buffer flow channel 321 is located, nucleic acid(s) in the sample solution is (are) attached to the magnetic beads, and the rest of the solution enters the waste liquid tank 35 as a waste solution.

Step S22 includes introducing a binding solution into the microfluidic channel through the binding solution inlet 332, withdrawing the magnetic field in the buffer area where the first buffer flow channel 321 is located, and applying a magnetic field to the buffer area where the second buffer flow channel 322 is located; and introducing air into the first venthole 335, such that the binding solution flows into the second buffer flow channel 322 through the first buffer flow channel 321, in the process, the magnetic beads are resuspended by the binding solution and then are adsorbed in the second buffer flow channel 322, and the waste solution (or liquid) without interacting with the magnetic beads enters the waste liquid tank 35.

Step S23 includes introducing a cleaning solution into the microfluidic channel through the cleaning solution inlet 333, withdrawing the magnetic fields in the buffer areas where the two buffer flow channels 32 are located, and introducing air into the second venthole 336, such that the cleaning solution flows from the second buffer flow channel 322 to the first buffer flow channel 321, the magnetic beads are resuspended by the cleaning solution when flowing through the second buffer flow channel 322. Further, air is introduced into the first venthole 335 when the solution reaches the first buffer flow channel 321 but does not flow out of the first buffer flow channel 321, keeping the withdrawing of the magnetic field in the buffer area where the first buffer flow channel 321 is located, and applying a magnetic field to the buffer area where the second buffer flow channel 322 is located, such that the magnetic beads are adsorbed in the second buffer flow channel 322, and the waste solution (or liquid) flows into the waste liquid tank 35.

Here, step S23 may be performed once or repeatedly for a plurality of times.

Step S24 includes keeping the applying of the magnetic field to the buffer area where the second buffer flow channel 322 is located, keeping the introducing of air to the first venthole 335, and keeping the second venthole 336 open, thereby ensuring that no liquid remains in the whole channel; and evaporating the residual liquid on the magnetic beads.

Step S25 includes introducing an eluent into the microfluidic channel through the eluent inlet 334, withdrawing the magnetic field in the buffer area where the second buffer flow channel 322 is located, applying a magnetic field to the buffer area where the first buffer flow channel 321 is located, and introducing air into the second venthole 336, such that the eluent flows into the first buffer flow channel 321 through the second buffer flow channel 322, and in this process, the magnetic beads are resuspended by the eluent.

Step S26 includes applying a magnetic field to at least one of the first buffer flow channel 321 and the second buffer flow channel 322 (for example, applying a magnetic field to both of the first buffer flow channel 321 and the second buffer flow channel 322) when the magnetic beads enter the first buffer flow channel 321, such that the magnetic beads are adsorbed. The film 39 covering the sample outlet 337 is mechanically broken, and air is continuously supplied to the first venthole 335 and the second venthole 336, such that a nucleic acid solution is discharged from the sample outlet 337, thereby completing the extraction of nucleic acid(s).

FIG. 5 is a top view of a microfluidic chip according to further embodiments of the present disclosure. The microfluidic chip shown in FIG. 5 is similar to that shown in FIG. 4A, and only the differences therebetween will be described below.

In FIG. 5, the plurality of openings 33 include not only the first venthole 335, the second venthole 336, the sample inlet 331, the binding solution inlet 332, the eluent inlet 334, the cleaning solution inlet 333 and the sample outlet 337, but also a waste liquid outlet 338 in communication with the second portion 312 of the primary flow channel 31 through a corresponding branch flow channel 34. In FIG. 5, the waste liquid outlet 338 is used for discharging a waste liquid (or a waste solution) out of the microfluidic channel without providing the waste liquid tank 35. Since the waste liquid can be discharged from the microfluidic chip in time, the microfluidic chip from which the waste liquid is discharged through the waste liquid outlet 338 is more advantageous to reuse of the microfluidic chip than the microfluidic chip with the waste liquid tank 35.

A nucleic acid extraction process by using the microfluidic chip shown in FIG. 5 will be described below, in where the two buffer flow channels 32 are respectively referred to as a first buffer flow channel 321 and a second buffer flow channel 322. The nucleic acid extraction process includes the following steps S31 to S36.

Step S31 includes applying a magnetic field to the buffer area where the first buffer flow channel 321 is located, but not applying a magnetic field to the buffer area where the second buffer flow channel 322 is located; introducing a mixed solution of a sample solution, a sample lysis solution and magnetic beads into the microfluidic channel through the sample inlet 331, opening the sample outlet 337, and introducing air into the first venthole 335, such that the mixed solution flows into the first buffer flow channel 321, the magnetic beads in the mixed solution are adsorbed in the buffer area where the first buffer flow channel 321 is located, nucleic acid(s) in the sample solution is (are) attached to the magnetic beads, and the rest of the solution is discharged from the waste liquid outlet 338 as a waste solution.

Step S32 includes introducing a binding solution into the microfluidic channel through the binding solution inlet 332, withdrawing the magnetic field in the buffer area where the first buffer flow channel 321 is located, and applying a magnetic field to the buffer area where the second buffer flow channel 322 is located; and introducing air into the first venthole 335, such that the binding solution flows into the second buffer flow channel 322 through the first buffer flow channel 321, in the process, the magnetic beads are resuspended by the binding solution and then are adsorbed in the second buffer flow channel 322, and the waste solution without interacting with the magnetic beads is discharged from the waste liquid outlet 338.

Step S33 includes introducing a cleaning solution into the microfluidic channel through the cleaning solution inlet 333, withdrawing the magnetic fields in the buffer areas where the two buffer flow channels 32 are located, and introducing air into the second venthole 336, such that the cleaning solution flows from the second buffer flow channel 322 to the first buffer flow channel 321, the magnetic beads are resuspended by the cleaning solution when the cleaning solution flows through the second buffer flow channel 322. Further, air is introduced into the first venthole 335 when the solution reaches the first buffer flow channel 321 but does not flow out of the first buffer flow channel 321, the magnetic field in the buffer area where the first buffer flow channel 321 is located is kept to be withdrawn, and a magnetic field is applied to the buffer area where the second buffer flow channel 322 is located, such that the magnetic beads are adsorbed in the second buffer flow channel 322, and the waste solution is discharged from the waste liquid outlet 338.

Here, step S33 may be performed once or repeatedly for a plurality of times.

Step S34 includes keeping the applying of the magnetic field to the buffer area where the second buffer flow channel 322 is located, keeping the introducing of air to the first venthole 335, and keeping the second venthole 336 open, thereby ensuring that no liquid remains in the whole channel and evaporating the residual liquid on the magnetic beads.

Step S35 includes introducing an eluent into the microfluidic channel through the eluent inlet 334, withdrawing the magnetic field in the buffer area where the second buffer flow channel 322 is located, applying a magnetic field to the buffer area where the first buffer flow channel 321 is located, and introducing air into the second venthole 336, such that the eluent flows into the first buffer flow channel 321 through the second buffer flow channel 322, and in this process, the magnetic beads are resuspended by the eluent.

Step S36 includes applying a magnetic field to at least one of the first buffer flow channel 321 and the second buffer flow channel 322 (for example, applying a magnetic field to both of the first buffer flow channel 321 and the second buffer flow channel 322) when the magnetic beads enter the first buffer flow channel 321, such that the magnetic beads are adsorbed. The film 39 covering the sample outlet 337 is mechanically broken and air is continuously supplied to the first venthole 335 and the second venthole 336, thereby discharging a nucleic acid solution from the sample outlet 337.

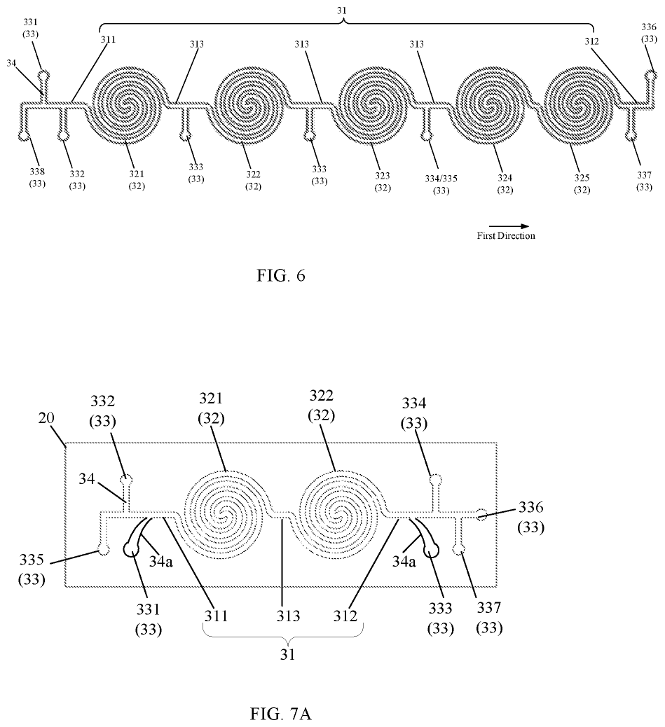

FIG. 6 is a top view of a microfluidic chip according to further embodiments of the present disclosure. The microfluidic chip shown in FIG. 6 is similar to the microfluidic chip shown in FIG. 4A, except for the arrangement of the openings 33 and the number of buffer flow channels 32. Only the differences between the microfluidic chips respectively shown in FIG. 6 and FIG. 4A will be described below.

In FIG. 6, the number of the buffer flow channels 32 is greater than two, for example, is five as shown in FIG. 6, but apparently, the number of the buffer flow channels 32 may be other numbers, for example, 4, 6, and so on. The plurality of buffer flow channels 32 are sequentially arranged in the first direction, and are located between the first portion 311 and the second portion 312 of the primary flow channel 31. At least adjacent two of the buffer flow channels 32 are in communication with each other through a connection portion 313 of the primary flow channel 31. For example, every adjacent two of the buffer flow channels 32 are in communication with each other through a connection portion 313, alternatively, every adjacent two of four buffer flow channels 32 are in communication with each other through a connection portion 313.

In the microfluidic chip shown in FIG. 6, the plurality of openings 33 optionally include the first venthole 335, the second venthole 336, the sample inlet 331, the binding solution inlet 332, the cleaning solution inlet 333, the eluent inlet 334, the waste liquid outlet 338, and the sample outlet 337. The waste liquid outlet 338, the sample inlet 331, and the binding solution inlet 332 are all in communication with the first portion 311 of the primary flow channel 31. The second venthole 336 and the sample outlet 337 are both in communication with the second portion 312. The cleaning solution inlet 333 and the eluent inlet 334 are in communication with connection portions 313, respectively, and the cleaning solution inlet 333 and the first venthole 335 are in communication with different connection portions 313, respectively.

In an example, as shown in FIG. 6, the number of the buffer flow channels 32 is five, and the five buffer flow channels 32 are respectively referred to as a first buffer flow channel 321, a second buffer flow channel 322, a third buffer flow channel 323, a fourth buffer flow channel 324, and a fifth buffer flow channel 325. The first buffer flow channel 321 is in communication with the first portion 311, and the fifth buffer flow channel 325 is in communication with the second portion 312. The first buffer flow channel 321 and the second buffer flow channel 322 are in communication with each other through a connection portion 313, and the second buffer flow channel 322 and the third buffer flow channel 323 are in communication with each other through a connection portion 313. Further, the third buffer flow channel 323 and the fourth buffer flow channel 324 are in communication with each other through a connection portion 313.

For example, the waste liquid outlet 338, the sample inlet 331, and the binding solution inlet 332 are all in communication with the first portion 311 of the primary flow channel 31 through the respective branch flow channels 34. The second venthole 336 and the sample outlet 337 are both in communication with the second portion 312 of the primary flow channel 31 through the respective branch flow channels 34. There are two cleaning solution inlets 333, one of which is in communication with the connection portion 313 between the first buffer flow channel 321 and the second buffer flow channel 322 through the corresponding branch flow channel 34, and the other is in communication with the connection portion 313 between the second buffer flow channel 322 and the third buffer flow channel 323 through the corresponding branch flow channel 34. The first venthole 335 and the eluent inlet 334 have a one-piece structure, i.e., one of the openings 33 serves as both the first venthole 335 and eluent inlet 334. The first venthole 335 is in communication with the connection portion 313 between the third buffer flow channel 323 and the fourth buffer flow channel 324 through the corresponding branch flow channel 34.

In the microfluidic chip shown in FIG. 6, the plurality of branch flow channels 34 are distributed on the upper and lower sides of the primary flow channel 31. However, an embodiment of the present disclosure does not limit which ones among the plurality of branch flow channels 34 are on the upper side of the primary flow channel 31 and which ones of the plurality of branch flow channels 34 are on the lower side of the primary flow channel 31. Alternatively, the plurality of branch flow channels 34 may be distributed on a same side of the primary flow channel 31, for example, on the upper side, or on the lower side.

In addition, it should be note that although it is illustrated in FIG. 6 that each opening 33 is in communication with the primary flow channel 31 through the corresponding branch flow channel 34, the present disclosure is not limited thereto. For example, no branch flow channel 34 may be provided between the waste liquid outlet 338 and the primary flow channel 31, and an orthogonal projection of the waste liquid outlet 338 on the first substrate 10 may be positioned on the primary flow channel 31. Further, no branch flow channel 34 may be provided between the second venthole 336 and the primary flow channel 31, and an orthogonal projection of the second venthole 336 on the first substrate 10 may be positioned on the primary flow channel 31.

In the microfluidic chip shown in FIG. 6, by providing a greater number of buffer flow channels 32 and making some of the reagent inlets and the connection portion 313 between adjacent two buffer flow channels 32 be in communication with each other, it is possible to prevent the buffer flow channels 32 from being contaminated by a waste liquid, as will be described later. A nucleic acid extraction process by using the microfluidic chip shown in FIG. 6 will be described below, in which the two cleaning solution inlets 333 are respectively referred to as a first cleaning solution inlet 333 and a second cleaning solution inlet 333. The nucleic acid extraction process includes the following steps S41 to S46.

Step S41 includes applying a magnetic field to the buffer area where the first buffer flow channel 321 is located, and applying no magnetic field to the buffer area where any one of the other buffer flow channels 32 is located; and introducing a mixed solution of a sample solution, a sample lysis solution, and magnetic beads into the microfluidic channel through the sample inlet 331, and closing the waste liquid outlet 338, such that the mixed solution flows rightward after entering the microfluidic channel, the magnetic beads are adsorbed in the first buffer flow channel 321 due to the magnetic field, and nucleic acid(s) in the sample solution is (are) attached to the magnetic beads. After the mixed solution is introduced into the sample inlet 331, the waste liquid outlet 338 is opened, and air is introduced into the second venthole 336 to discharge a waste liquid from the waste liquid outlet 338, and then the waste liquid outlet 338 is closed.

Step S42 includes introducing a binding solution into the microfluidic channel through the binding solution inlet 332, applying a magnetic field to the buffer area where the second buffer flow channel 322 is located, and withdrawing the magnetic field in the buffer area where any one of the rest of the buffer flow channels 32 is located. After entering the microfluidic channel, the binding solution flows to the right, the magnetic beads are resuspended by the binding solution when the binding solution passes through the first buffer flow channel 321, then the magnetic beads are flushed (or moved) to the second buffer flow channel 322, such that the magnetic beads are adsorbed in the second buffer flow channel 322. After the binding solution is introduced through the binding solution inlet 332, the waste liquid outlet 338 is opened, and air is introduced into the second venthole 336 to discharge a waste liquid from the waste liquid outlet 338, and then the waste liquid outlet 338 is closed.

Step S43 includes introducing a cleaning solution into the microfluidic channel through the first cleaning solution inlet 333, applying a magnetic field to the buffer area where the third buffer flow channel 323 is located, and withdrawing the magnetic field in the buffer area where any one of the remaining buffer flow channels 32 is located. After entering the microfluidic channel, the cleaning solution flows to the right, the magnetic beads are resuspended by the cleaning solution when the cleaning solution passes through the second buffer flow channel 322, and then the magnetic beads are flushed to the third buffer flow channel 323, such that the magnetic beads are adsorbed in the third buffer flow channel 323. After the cleaning solution is introduced into the first cleaning solution inlet 333, the waste liquid outlet 338 is opened, and air is introduced into the second venthole 336 to discharge a waste liquid from the waste liquid outlet 338, and then the waste liquid outlet 338 is closed.

Step S44 includes introducing a cleaning solution into the microfluidic channel through the second cleaning solution inlet 333, applying a magnetic field to the buffer area where the fourth buffer flow channel 324 is located, and withdrawing the magnetic field in the buffer area where any one of the remaining buffer flow channels 32 is located. After entering the microfluidic channel, the cleaning solution flows to the right, and the magnetic beads are resuspended by the cleaning solution when the cleaning solution passes through the third buffer flow channel 323, and the magnetic beads are flushed to the fourth buffer flow channel 324, such that the magnetic beads are adsorbed in the fourth buffer flow channel 324. After the cleaning solution is introduced into the second cleaning solution inlet 333, the waste liquid outlet 338 is opened, and air is introduced into the second venthole 336, thereby discharging a waste liquid from the waste liquid outlet 338.

Step S45 includes opening the waste liquid outlet 338, and introducing air into the second venthole 336 for a period of time (e.g., for 5 min), to ensure that the entire channel is free of liquid and to evaporate residual liquid from the magnetic beads. Then, the waste liquid outlet 338 is closed.

Step S46 includes introducing an eluent into the microfluidic channel through the eluent inlet 334, applying a magnetic field to the buffer area where the fifth buffer flow channel 325 is located, and withdrawing the magnetic field in the buffer area where any one of the remaining buffer flow channels 32 is located. The eluent flows to the right after entering the microfluidic channel, and the magnetic beads are resuspended by the eluent when the eluent passes through the fourth buffer flow channel 324, and the magnetic beads are flushed to the fifth buffer flow channel 325, such that the magnetic beads are adsorbed in the fifth buffer flow channel 325. After the elution solution is introduced, the second venthole 336 is closed, the sample outlet 337 is opened, and air is introduced into the first venthole 335, such that the sample solution is discharged from the sample outlet 337, thereby completing the nucleic acid extraction.

It can be seen that when the microfluidic chip shown in FIG. 6 is used for nucleic acid extraction, after each time a reagent enters a certain buffer flow channel 32 to be in sufficient contact with the magnetic beads, the generated waste liquid flows leftwards to the waste liquid outlet, whereas the next introduced reagent flows to the buffer flow channel 32 further to the right, such that the contact of the next introduced reagent with the residual waste liquid of the previous reagent is reduced, and the nucleic acid extraction efficiency is improved.

FIG. 7A is a top view of a microfluidic chip according to further embodiments of the present disclosure, and FIG. 7B is a schematic diagram showing a connection between one of the branch flow channels and the primary flow channel shown in FIG. 7A. The microfluidic chip shown in FIG. 7A is similar to the microfluidic chip shown in FIG. 5 except that each of the branch flow channels 34 in FIG. 5 may have a shape of a straight line perpendicular to the primary flow channel 31, and widths of portions of each branch flow channel 34 in FIG. 5 may be the same or approximately the same as each other; whereas at least one of the branch flow channels 34 in FIG. 7A may have a shape of a straight line inclined to the primary flow channel 31, or may even have a shape of a non-straight line.

As shown in FIG. 7A and FIG. 7B, each branch flow channel 34 may have a first opening H1 and a second opening H2, where the first opening H1 is in communication with the primary flow channel 31, and the second opening H2 is in communication with one of the inlets. An orthogonal projection of each branch flow channel 34 on the first substrate 10 has a first edge and a second edge, both of which are connected between the first opening H1 and the second opening H2. At least one of the branch flow channels 34 is a tapered flow channel 34a, and a diameter (or caliber) of the first opening H1 of the tapered flow channel 34a is less than a diameter (or caliber) of the second opening H2 of the tapered flow channel 34a. In this case, a solution in this tapered flow channel 34a can flow into the primary flow channel 31 from the second opening H2, but the solution in the primary flow channel 31 is subjected to a large flow resistance when flowing back to this tapered flow channel 34a, such that the solution is not easy to flow back to tapered flow channel 34a, thereby reducing or avoiding the flowing back of the solution.

The primary flow channel 31 includes a first portion 311 and a second portion 312, and the plurality of buffer flow channels 32 are located between the first portion 311 and the second portion 312. Further, the first opening H1 of the tapered flow channel 34a is in communication with the first portion 311 or the second portion 312. That is, at least one branch flow channel 34 in communication with the first portion 311 or the second portion 312 may be configured as the tapered flow channel 34a.

In an example, the branch flow channel 34 in communication with the sample inlet 331 is configured as the tapered flow channel, and in the first direction, a distance from the first opening H1 of the tapered flow channel 34a to a buffer flow channel 32 is less than a distance from the second opening H2 of the tapered flow channel 34a to the buffer flow channel 32. In this case, when a binding solution is introduced into the primary flow channel 31 through the binding solution inlet 332, the binding solution flows rightward to the buffer flow channel 32, and when the binding solution passes through the first opening H1 of the branch flow channel 34 (i.e., the tapered flow channel 34a) in communication with the sample inlet 331, the binding solution hardly flows into the tapered flow channel 34a through the first opening H1.

In another example, the branch flow channel 34 in communication with the cleaning solution inlet 333 is configured as the tapered flow channel 34a, and in the first direction, a distance from the first opening H1 of the tapered flow channel 34a to an adjacent buffer flow channel 32 is less than a distance from the second opening H2 of the tapered flow channel 34a to the adjacent buffer flow channel 32. In this case, when an eluent is introduced into the primary flow channel 31 through the eluent inlet 334, the eluent flows leftwards to the buffer flow channel 32, and when the eluent passes through the first opening H1 of the branch flow channel 34 (i.e., the tapered flow channel 34a) in communication with the cleaning solution inlet 333, the eluent hardly flows into the tapered flow channel through the first opening H1.

Here, as shown in FIG. 7B, the first edge and the second edge of the tapered flow channel 34a are both arc-shaped, thereby further preventing a liquid in the primary flow channel 31 from flowing back into the tapered flow channel 34a.

It should be noted that the microfluidic chip shown in FIG. 7A is an improvement of the microfluidic chip shown in FIG. 5, and for the microfluidic chip according to any one of other embodiments, at least one of the branch flow channels 34 may also be configured as the tapered flow channel. Specifically, if the first portion 311 or the second portion 312 is provided with multiple branch flow channels 34 in communication with the reagent inlets, respectively, the branch flow channel 34 adjacent to a buffer flow channel 32 may be configured as the tapered flow channel. For example, in the microfluidic chip shown in FIG. 4A, the branch flow channel 34 in communication with the sample inlet 331 and the branch flow channel 34 in communication with the cleaning solution inlet 333 may be designed as shown in FIG. 7A. For another example, in the microfluidic chip shown in FIG. 6, the branch flow channels 34 in communication with the binding solution inlet 332 may be designed according to the branch flow channels 34 in communication with the sample inlet 331 in FIG. 7A, so as to prevent the solution introduced into the primary flow channels 31 through the sample inlet 331 in FIG. 6 from flowing back into the branch flow channel 34 in communication with the binding solution inlet 332.

Embodiments of the present disclosure further provide a nucleic acid extraction apparatus, which includes the microfluidic chip according to any one of the foregoing embodiments and a magnetic control device, where the magnetic control device is configured to apply a magnetic field to the buffer areas BA of the microfluidic chip independently. Here, to apply a magnetic field to the buffer areas BA of the microfluidic chip “independently” means that: the applying and the not applying a magnetic field to different buffer areas BA, respectively, are independent of each other.

In some embodiments, the nucleic acid extraction apparatus further includes a mounting frame (not shown), on which the microfluidic chip and the magnetic control device are disposed. The magnetic control device may be positioned above the microfluidic chip or below the microfluidic chip. Here, the term “above” refers to a side of the second substrate 20 distal to the first substrate 10, and the term “below” refers to a side of the first substrate 10 distal to the second substrate 20.

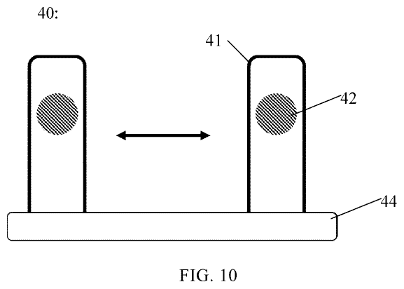

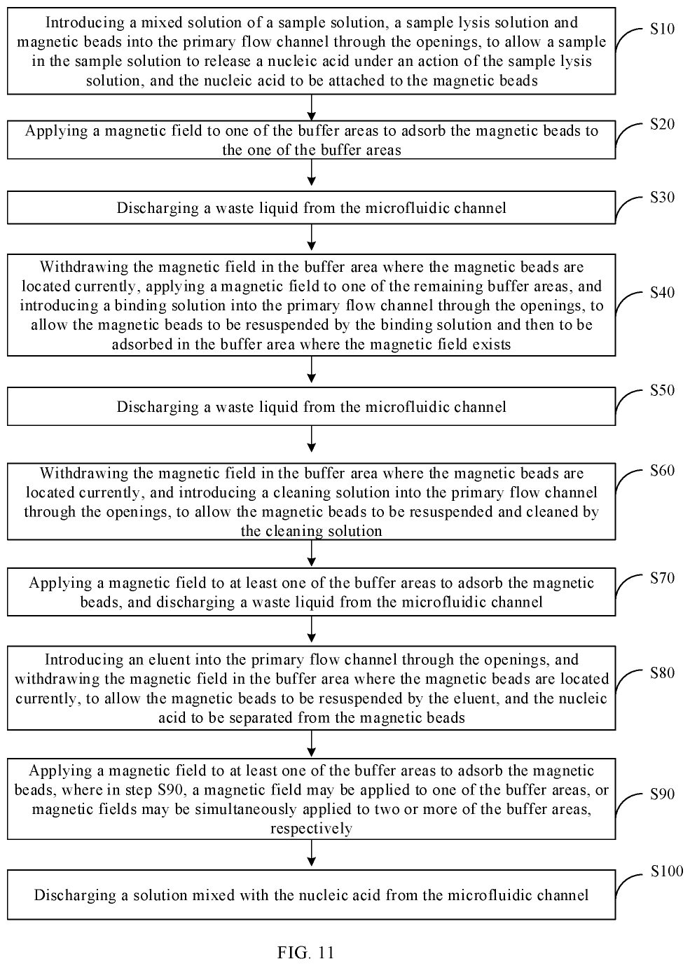

FIG. 8 is a top view of a magnetic control device according to some embodiments of the present disclosure, and FIG. 9 is a top view of a magnetic control structure according to other embodiments of the present disclosure. As shown in FIGS. 8 and 9, the magnetic control device includes a magnet mounting part 41, a magnet 42 and a rotating part 43, where it is possible to provide one or more magnets 42, and the one or more magnets 42 are arranged on the magnet mounting part 41. The rotating part 43 is connected to the magnet mounting part 41 and the mounting frame, and is configured to drive the magnet mounting part 41 to rotate around an axis of the rotating part 43, so as to make the magnet 42 move between any two of an initial position and positions respectively opposite to the buffer areas BA. The initial position and any one of the buffer areas BA do not overlap each other in a thickness direction of the microfluidic chip. That is, an orthogonal projection of the initial position on a reference plane parallel to the microfluidic chip does not overlap an orthogonal projection of any one of the buffer areas BA on the reference plane.

Here, the rotating part 43 may include a gear, a rotating shaft, and/or another rotating structure.

In some embodiments, as shown in FIG. 8, there is provided one magnet 42, and the magnet mounting part has a rectangular structure and includes a first end and a second end arranged along a lengthwise direction thereof. Further, the rotating part 43 is connected to a middle position of the magnet mounting part, and the magnet 42 is disposed between the rotating part 43 and the first end. The “middle position” refers to a position at or near the midpoint between the first end and the second end.

In other embodiments, as shown in FIG. 9, there are provided a plurality of magnets 42, and the magnet mounting part has a shape of a circular disc. Further, the rotating part 43 is disposed at a center of the magnet mounting part. A peripheral portion of the magnet mounting part has a plurality of magnet installing areas and a plurality of vacant areas DA. Each magnet installing area is provided therein with a magnet 42. The plurality of magnet installing areas and the plurality of vacant areas DA are divided into at least one first area group, at least one second area group, and at least one third area group. Each first area group includes a magnet installing area and a vacant area DA that are located at both ends of a diameter of the magnet mounting part 41, respectively. Each second area group includes two magnet installing areas that are located at both ends of a diameter of the magnet mounting part 41, respectively. Each third area group includes two vacant areas DA that are located at both ends of a diameter of the magnet mounting part 41, respectively.

In a case where the number of the buffer flow channels 32 of the microfluidic chip is two, the magnet mounting part shown in FIG. 8 may be adopted, and in this case, by rotation of the magnet mounting part, a magnetic field may be applied to the buffer area where one of the buffer flow channels 32 is located, or no magnetic field may be applied to any one of the buffer flow channels 32.

In a case where the number of the buffer flow channels 32 of the microfluidic chip is two, the magnet mounting part shown in FIG. 9 may alternatively be adopted, and in this case, by rotation of the magnet mounting part, a magnetic field may be applied to the buffer area where one of the buffer flow channels 32 is located, or a magnetic field may be applied to the buffer areas where both of the buffer flow channels 32 are respectively located at the same time, or no magnetic field may be applied to any one of the buffer flow channels 32.

FIG. 10 is a top view of a magnetic control structure according to further embodiments of the present disclosure. A shown in FIG. 10, the magnetic control structure includes a guide rail 44, a magnet mounting part 41, and a magnet 42. The guide rail 44 extends along a direction in which the plurality of buffer areas BA of the microfluidic chip are arranged. The magnet mounting part 41 is arranged on the guide rail 44, and the magnet 42 is provided on the magnet mounting part 41. The magnet mounting part is configured to move along the guide rail 44 to move the magnet 42 between any two of the initial position and the positions respectively opposite to the buffer areas BA, where the initial position and any one of the buffer areas BA do not overlap each other in the thickness direction of the microfluidic chip.

In the case where the number of the buffer flow channels 32 of the microfluidic chip is greater than 2, the magnetic control device may adopt the structure shown in FIG. 10 to move the magnet mounting part as needed, so as to move the magnet 42 to the position of any one of the buffer flow channels 32. Alternatively, in FIG. 10, a plurality of magnet mounting parts 41 may be provided on the guide rail 44, such that magnetic fields can be applied to a plurality of buffer areas BA at the same time, respectively.

Here, in the magnetic control structure 40 shown in each of FIGS. 8 to 10, each magnet 42 may be a permanent magnet 42, and a shape of an orthogonal projection of each magnet 42 on the magnet mounting part 41 may be a circle. In an embodiment of the present disclosure, the movement of each magnet 42 is controlled by a mechanical manner, such that magnetic fields are applied to the buffer flow channels 32 independently. Compared with the use of an electromagnet, the magnetic control device according to an embodiment of the present disclosure has a lower cost, and compared with the electromagnet, the use of a permanent magnet does not generate heat, and thus, a bulky cooling assembly is not necessary, which is beneficial to simplifying the structure of the nucleic acid extraction apparatus.

Embodiments of the present disclosure further provide a nucleic acid extraction method, which may be applied to the microfluidic chip according to any one of the foregoing embodiments. FIG. 11 is a schematic diagram of the nucleic acid extraction method according to some embodiments of the present disclosure. As shown in FIG. 11, the nucleic acid extraction method includes the following steps S10 to S100.

Step S10 includes introducing a mixed solution of a sample solution, a sample lysis solution and magnetic beads into the primary flow channel through a corresponding one of the openings, to allow a sample in the sample solution to release nucleic acid(s) under the action of the sample lysis solution, and the nucleic acid(s) to be attached to the magnetic beads.

Step S20 includes applying a magnetic field to one of the buffer areas to adsorb the magnetic beads to the one of the buffer areas.

Step S30 includes discharging a waste liquid from the microfluidic channel.

Step S40 includes withdrawing the magnetic field in the buffer area where the magnetic beads are located currently, applying a magnetic field to one of the remaining buffer areas, and introducing a binding solution into the primary flow channel through a corresponding one of the openings, to allow the magnetic beads to be resuspended by the binding solution and then to be adsorbed in the buffer area where the magnetic field exists.

Step S50 includes discharging the waste liquid from the microfluidic channel.

Step S60 includes withdrawing the magnetic field in the buffer area where the magnetic beads are located currently, and introducing a cleaning solution into the primary flow channel through a corresponding one of the openings, to allow the magnetic beads to be resuspended and cleaned by the cleaning solution.

Step S70 includes applying a magnetic field to at least one of the buffer areas to adsorb the magnetic beads, and discharging the waste liquid from the microfluidic channel.

Step S80 includes introducing an eluent into the primary flow channel through a corresponding one of the openings, and withdrawing the magnetic field in the buffer area where the magnetic beads are located currently, to allow the magnetic beads to be resuspended by the eluent, and the nucleic acid(s) to be separated from the magnetic beads.

Step S90 includes applying a magnetic field to at least one of the buffer areas to adsorb the magnetic beads. In step S90, a magnetic field may be applied to one of the buffer areas, or magnetic fields may be simultaneously applied to two or more of the buffer areas, respectively.

Step S100 includes discharging a solution mixed with the nucleic acid(s) from the microfluidic channel.

In the case where the microfluidic chip has the first venthole and the second venthole according to any one of the foregoing embodiments, in the above steps, the discharging of the waste liquid from the microfluidic channel may be achieved by sucking air from (i.e., applying a negative pressure to) the first venthole or the second venthole, alternatively, by introducing air into the first venthole or the second venthole, which may be specifically determined according to the actual structure of the microfluidic chip.

In the case where the microfluidic chip is provided with the sample outlet, and the sample outlet is covered with the film, step S90 may include mechanically breaking the film, and sucking air from or introducing air into one of the first venthole and the second venthole, to allow the solution mixed with the nucleic acid(s) to be discharged from the microfluidic chip through the sample outlet.

In some embodiments, before step S80, the nucleic acid extraction method further includes sucking air from or introducing air into one of the first venthole and the second venthole, to remove the waste liquid remaining in the microfluidic channel.

The nucleic acid extraction process by using each of the microfluidic chip according to any one of the foregoing embodiments has been described above, and detailed description thereof is not repeated here.

It should be understood that the above embodiments are merely exemplary embodiments employed to illustrate the principles of the present disclosure, and the present disclosure is not limited thereto. It will be apparent to one of ordinary skill in the art that various changes and modifications may be made therein without departing from the spirit and scope of the present disclosure, and such changes and modifications also fall within the scope of the present disclosure.

1. A microfluidic chip, configured to extract a nucleic acid, the microfluidic chip comprising:
a first substrate having at least two buffer areas configured to cooperate with a magnet, and being provided with a microfluidic channel, wherein the microfluidic channel comprises a primary flow channel and a plurality of buffer flow channels connected in series to the primary flow channel, the buffer flow channels are in the buffer areas, different buffer flow channels are in different buffer areas, respectively, and each of the buffer flow channels has a double-spiral shape; and
a second substrate opposite to the first substrate;
wherein the microfluidic chip has a plurality of openings in communication with the primary flow channel.

2. The microfluidic chip according to claim 1, wherein the primary flow channel extends in a first direction, the microfluidic channel further comprises branch flow channels located on a side of the primary flow channel in a second direction, and the second direction intersects the first direction; and
at least one of the openings is in communication with the primary flow channel through at least one of the branch flow channels, respectively.

3. The microfluidic chip according to claim 2, wherein the plurality of openings comprise a first venthole, a second venthole, a sample inlet, a binding solution inlet, a cleaning solution inlet, and an eluent inlet, and each of the sample inlet, the binding solution inlet, the cleaning solution inlet, and the eluent inlet is in communication with the primary flow channel through a corresponding one of the branch flow channels.

4. The microfluidic chip according to claim 3, wherein each of the branch flow channels in communication with the sample inlet, the binding solution inlet, the cleaning solution inlet and the eluent inlet, respectively, is provided with a first valve.

5. The microfluidic chip according to claim 3, wherein the branch flow channel in communication with the sample inlet, the branch flow channel in communication with the binding solution inlet, the branch flow channel in communication with the cleaning solution inlet, and the branch flow channel in communication with the eluent inlet are sequentially arranged along the first direction, the branch flow channel in communication with the binding solution inlet and the branch flow channel in communication with the sample inlet are both in communication with the primary flow channel through a first combining flow channel, and the branch flow channel in communication with the cleaning solution inlet and the branch flow channel in communication with the eluent inlet are both in communication with the primary flow channel through a second combining flow channel.

6. The microfluidic chip according to claim 5, wherein the microfluidic channel further comprises a connection flow channel connected between the first combining flow channel and the second combining flow channel, and the connection flow channel has a second valve disposed thereon.

7. The microfluidic chip according to claim 3, wherein the first substrate further has a waste liquid tank disposed thereon, and the second venthole is in communication with the primary flow channel via the waste liquid tank; and
wherein the waste liquid tank protrudes in a direction away from the second substrate.

8. (canceled)

9. The microfluidic chip according to claim 3, wherein the plurality of openings further comprises a waste fluid outlet in communication with the primary flow channel.

10. The microfluidic chip according to claim 1, wherein the plurality of openings further comprises a sample outlet in communication with the primary flow channel; and
the sample outlet penetrates through the first substrate, and an end of the sample outlet distal to the second substrate is covered with a film.

11. The microfluidic chip according to claim 3, wherein the primary flow channel comprises a first portion and a second portion, the plurality of buffer flow channels are all located between the first portion and the second portion;
the first venthole, the sample inlet, and the binding solution inlet are all in communication with the first portion, and the second venthole, the cleaning solution inlet, and the eluent inlet are all in communication with the second portion;
wherein the first portion of the primary flow channel has a first liquid inlet and a second liquid inlet, the second portion of the primary flow channel has a third liquid inlet and a fourth liquid inlet, the sample inlet is in communication with the first liquid inlet through a respective branch flow channel, the binding solution inlet is in communication with the second liquid inlet through a respective branch flow channel, the cleaning solution inlet is in communication with the third liquid inlet through a respective branch flow channel, and the eluent inlet is in communication with the fourth liquid inlet through a respective branch flow channel; and
the first liquid inlet is on a side of the second liquid inlet proximal to the buffer flow channels, and the third liquid inlet is on a side of the fourth liquid inlet proximal to the buffer flow channels.

12. (canceled)

13. The microfluidic chip according to claim 3, wherein the microfluidic channel comprises at least three buffer flow channels arranged in the first direction, the primary flow channel comprises a first portion, a second portion, and connection portions, the at least three buffer flow channels are all located between the first portion and the second portion, and at least two of the buffer flow channels are in communication with each other through the connection portions, respectively;
the plurality of openings further comprise a waste liquid outlet and a sample outlet, the waste liquid outlet, the sample inlet, and the binding fluid inlet are all in communication with the first portion, the second venthole and the sample outlet are both in communication with the second portion, the cleaning solution inlet and the eluent inlet are in communication with the connection portions, and the cleaning solution inlet and the eluent inlet are in communication with different connection portions, respectively; and
wherein the at least three buffer flow channels comprise a first buffer flow channel, a second buffer flow channel, a third buffer flow channel, a fourth buffer flow channel, and a fifth buffer flow channel which are arranged in sequence along the first direction, the first buffer flow channel is in communication with the first portion, the fifth buffer flow channel is in communication with the second portion, and the first buffer flow channel and the second buffer flow channel are in communication with each other, the second buffer flow channel and the third buffer flow channel are in communication with each other, and the third buffer flow channel and the fourth buffer flow channel are in communication with each other, through the connection portions, respectively; and the microfluidic chip has two cleaning solution inlets, one of which is in communication with the connection portion between the first buffer flow channel and the second buffer flow channel through a respective branch flow channel, the other is in communication with the connection portion between the second buffer flow channel and the third buffer flow channel through a respective branch flow channel, and the eluent inlet is in communication with the connection portion between the third buffer flow channel and the fourth buffer flow channel through a respective branch flow channel;
or
wherein the eluent inlet and the first venthole have a one-piece structure.

14-15. (canceled)

16. The microfluidic chip according to claim 3, wherein each of the branch flow channels has a first opening and a second opening, the first opening is in communication with the primary flow channel, the second opening is in communication with one of the inlets, at least one of the branch flow channels is a tapered flow channel, and a diameter of the first opening of the tapered flow channel is less than a diameter of the second opening of the tapered flow channel;
wherein the primary flow channel comprises a first portion and a second portion, the plurality of buffer flow channels are all located between the first portion and the second portion, and the first opening of the tapered flow channel is in communication with the first portion or the second portion;
in the first direction, a distance between the first opening of the tapered flow channel and any one of the buffer flow channels is less than a distance between the second opening of the tapered flow channel and the any one of the buffer flow channels; and
wherein an orthogonal projection of each of the branch flow channels on the first substrate has a first edge and a second edge, and each of the first edge and the second edge of the tapered flow channel is arc-shaped.

17-18. (canceled)

19. The microfluidic chip according to claim 1, wherein a width of any portion of each of the buffer flow channels is substantially the same as a width of the primary flow channel.

20. A nucleic acid extraction apparatus, comprising the microfluidic chip according to claim 1, and a magnetic control device for applying a magnetic field to the buffer areas of the microfluidic chip independently.

21. The nucleic acid extraction apparatus according to claim 20, further comprising a mounting frame on which the microfluidic chip and the magnetic control device are both mounted, wherein
the magnetic control device comprises:
a magnet mounting part;
a magnet on the magnet mounting part; and
a rotating part connected to the magnet mounting part and the mounting frame, and configured to drive the magnet mounting part to rotate around an axis of the rotating part, so as to make the magnet move between any two of an initial position and positions respectively opposite to the buffer areas, wherein the initial position and any one of the buffer areas do not overlap each other in a thickness direction of the microfluidic chip; and
wherein
the magnetic control device comprises one magnet, the magnet mounting part is a rectangular structure and comprises a first end and a second end along a lengthwise direction of the magnet mounting part, the rotating part is connected to a middle position of the magnet mounting part, and the magnet is arranged between the rotating part and the first end;
or
the magnetic control device comprises a plurality of magnets, and the magnet mounting part has a shape of a circular disc, the rotating part is disposed at a center of the magnet mounting part, a peripheral portion of the magnet mounting part has a plurality of magnet installing areas and a plurality of vacant areas, each of the magnet installing areas is provided therein with one of the magnets, the plurality of magnet installing areas and the plurality of vacant areas are divided into at least one first area group, at least one second area group, and at least one third area group, each first area group comprises one of the magnet installing areas and one of the vacant areas both of which are located at both ends of a diameter of the magnet mounting part, respectively, each second area group comprises two of the magnet installing areas both of which are located at both ends of a diameter of the magnet mounting part, respectively, and each third area group comprises two of the vacant areas both of which are located at both ends of a diameter of the magnet mounting part, respectively.

22. (canceled)

23. The nucleic acid extraction apparatus according to claim 20, further comprising a mounting frame on which the microfluidic chip and the magnetic control device are both mounted, wherein
the magnetic control device comprises:
a guide rail extending in a direction in which the buffer areas of the microfluidic chip are arranged;
a magnet mounting part on the guide rail; and
a magnet on the magnet mounting part;
wherein the magnet mounting part is configured to move along the guide rail to make the magnet move between any two of an initial position and positions respectively opposite to the buffer areas, and the initial position and any one of the buffer areas do not overlap each other in a thickness direction of the microfluidic chip.

24. A nucleic acid extraction method for the microfluidic chip according to claim 1, the nucleic acid extraction method comprising:
introducing a mixed solution of a sample solution, a sample lysis solution and magnetic beads into the primary flow channel through the openings, to allow a sample in the sample solution to release a nucleic acid under an action of the sample lysis solution, and the nucleic acid to be attached to the magnetic beads;
applying a magnetic field to one of the buffer areas to adsorb the magnetic beads to the one of the buffer areas;
discharging a waste liquid from the microfluidic channel;
withdrawing the magnetic field in the buffer area where the magnetic beads are located currently, applying a magnetic field to one of the remaining buffer areas, and introducing a binding solution into the primary flow channel through the openings, to allow the magnetic beads to be resuspended by the binding solution and then to be adsorbed in the buffer area where the magnetic field exists;
discharging a waste liquid from the microfluidic channel;
withdrawing the magnetic field in the buffer area where the magnetic beads are located currently, and introducing a cleaning solution into the primary flow channel through the openings, to allow the magnetic beads to be resuspended and cleaned by the cleaning solution;
applying a magnetic field to at least one of the buffer areas to adsorb the magnetic beads, and discharging a waste liquid from the microfluidic channel;
introducing an eluent into the primary flow channel through the openings, and withdrawing the magnetic field in the buffer area where the magnetic beads are located currently, to allow the magnetic beads to be resuspended by the eluent, and the nucleic acid to be separated from the magnetic beads;
applying a magnetic field to at least one of the buffer areas to adsorb the magnetic beads; and
discharging a solution mixed with the nucleic acid from the microfluidic channel.

25. The nucleic acid extraction method according to claim 24, wherein the primary flow channel extends in a first direction, the microfluidic channel further comprises branch flow channels located on a side of the primary flow channel in a second direction, and the second direction intersects the first direction;
at least one of the openings is in communication with the primary flow channel through at least one of the branch flow channels, respectively;
the plurality of openings comprise a first venthole, a second venthole, a sample inlet, a binding solution inlet, a cleaning solution inlet, and an eluent inlet, and each of the sample inlet, the binding solution inlet, the cleaning solution inlet, and the eluent inlet is in communication with the primary flow channel through a corresponding one of the branch flow channels;
the mixed solution is introduced into the primary flow channel through the sample inlet, the binding solution is introduced into the primary flow channel through the binding solution inlet, the cleaning solution is introduced into the primary flow channel through the cleaning solution inlet, and the eluent is introduced into the primary flow channel through the eluent inlet; and
the waste liquid is discharged each time by sucking air from or introducing air into one of the first venthole and the second venthole.

26. The nucleic acid extraction method according to claim 24, wherein the plurality of openings further comprises a sample outlet in communication with the primary flow channel, the sample outlet penetrates through the first substrate, and an end of the sample outlet distal to the second substrate is covered with a film;
the discharging the solution mixed with the nucleic acid from the microfluidic channel, comprises:
breaking the film mechanically, and sucking air from or introducing air into one of the first venthole and the second venthole, to allow the solution mixed with the nucleic acid to be discharged from the microfluidic chip through the sample outlet.

27. The nucleic acid extraction method according to claim 24, wherein prior to the introducing the eluent into the primary flow channel through the openings, the nucleic acid extraction method further comprises:
sucking air from or introducing air into one of the first venthole and the second venthole, to remove the waste liquid remaining in the microfluidic channel.
